(19) United States
(12) Patent Application Publication
(10) Pub. No.: US 2008/0194451 A1
(43) Pub. Date: Aug. 14, 2008

(54) 1-Bromopropane Having Low Acidity

(75) Inventors: Vincent Luc Mutterer, Rosenau (FR); Jean-Philippe Tritz, Rixheim (FR)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/912,458

(22) PCT Filed: May 1, 2006
(86) PCT No.: PCT/US2006/016669

§ 371 Date: Oct. 24, 2007

Related U.S. Application Data
(60) Provisional application No. 60/677,076, filed on May 3, 2005.

(51) Int. Cl. C11D 17/00; C07C 17/38

(52) U.S. Cl. 510/412; 570/262

(57) ABSTRACT

This invention provides a process which comprises mixing permanganate with a 1-bromopropane product mixture to form a permanganate-containing 1-bromopropane product mixture, and recovering a purified 1-bromopropane product from said permanganate-containing 1-bromopropane product mixture. This invention also provides a process for preparing a 1-bromopropane product mixture from 1-propanol and hydrogen bromide, characterized by mixing permanganate with the 1-bromopropane product mixture to form a permanganate-containing 1-bromopropane product mixture and recovering a purified 1-bromopropane product from said permanganate-containing 1-bromopropane product mixture.

### TECHNICAL FIELD

This invention relates to 1-bromopropane products having a reduced tendency for acid formation during storage, and to processes for producing such products.

### BACKGROUND

1-Bromopropane (also referred to as n-propyl bromide or propyl bromide) can be used as a degreasing agent, especially for degreasing metal parts, as well as in cleaning solutions for electrical circuit board production. In such applications, the presence of certain impurities in the 1-bromopropane is undesirable because the impurities have a detrimental effect on the substrates with which the 1-bromopropane is brought into contact. When manufactured, an initial 1-bromopropane product mixture contains impurities, including one or more of 2-bromopropane(isopropyl bromide), 1,2-dibromopropane, monobromoacetone, 1-propanol, propionaldehyde, propionic acid, water, and hydrogen bromide (HBr). Some of these impurities can, over time, degrade to yield acids, such as HBr and propionic acid. Thus, trace amounts of HBr are often present in finished 1-bromopropane products; further, the acidity increases over time in a finished 1-bromopropane product. Acidic species are detrimental to the performance qualities of 1-bromopropane as they can corrode or discolor the substrates to which the 1-bromopropane is applied. The art has tried to address this problem by adding acid scavenging compounds, such as 1,2-epoxides to the 1-bromopropane. Metal passivators, such as nitromethane and 1,3-dioxolane, have also been used. The problem with such additives is that there is a cost associated with their use, and they are consumed as they act to reduce acid content in the 1-bromopropane product. The consumption rate of the additive(s) can be such that, after a period of time, there is no further additive to reduce acid presence in the 1-bromopropane product. In this condition, the acid content of the 1-bromopropane can increase to deleterious levels. Thus, a better method for minimizing the acidic impurities in 1-bromopropane is needed, particularly one that does not compromise performance of the 1-bromopropane.

### SUMMARY OF THE INVENTION

This invention provides finished 1-bromopropane products having a reduced tendency to produce acidic species overtime. In particular, the invention can provide finished 1-bromopropane products that meet the requirement of keeping the acidity below 10 ppm for at least 30 days at 60° C. without detracting from the performance of the finished 1-bromopropane product. Surprisingly, it has been found that contacting a 1-bromopropane product mixture with permanganate, an oxidant, reduces or prevents the formation of acidity in a finished 1-bromopropane product produced therefrom. In particular, when a 1-bromopropane product mixture is treated with permanganate pursuant to this invention, the acidity of the finished 1-bromopropane product is reduced (in comparison to a finished 1-bromopropane product not so treated), and often the acidity of a permanganate-treated finished 1-bromopropane product remains below 10 ppm for at least 30 days at 60° C. As used herein, the term “finished 1-bromopropane product” denotes a 1-bromopropane product mixture that has been treated with permanganate, had the permanganate removed therefrom, and optionally has been subjected to further purification. Similarly, the term “1-bromopropane product mixture” denotes generally 1-bromopropane containing one or more of the impurities that are formed therewith during the course of manufacture of 1-bromopropane. More specifically, the 1-bromopropane product mixtures used in the processes of this invention generally contain 1-bromopropane and one or more of the impurities that are formed therewith during the course of manufacture of 1-bromopropane; the impurities are as described above.

This invention relates in part to processes for removing acidic and/or acid-forming species from 1-bromopropane product mixtures irrespective of the method of preparation of the 1-bromopropane product mixture. The processes of the invention result in the reduction of acidic species and/or acid-forming species, including propanol, propionaldehyde, and propionic acid. For example, 1-bromopropane product mixtures formed from 1-propanol and HBr typically contain propanol, propionaldehyde, and propionic acid, and 1-bromopropane product mixtures formed from propene and HBr often contain propionic acid. Thus, practicing the processes of this invention upon 1-bromopropane product mixtures made by such processes is advantageous.

Pursuant to this invention, a 1-bromopropane product mixture is mixed with permanganate. After the mixing, purification steps are taken to reduce the content of the pennanganate oxidation products and unreacted permanganate in the 1-bromopropane product mixture. The finished 1-bromopropane product is obtained from the permanganate-contacted 1-bromopropane product mixture. If desired, conventional additives such as acid scavengers and metal passivators can be added to the finished 1-bromopropane products of this invention.

An embodiment of this invention is a process which comprises mixing permanganate with a 1-bromopropane product mixture to form a permanganate-containing 1-bromopropane product mixture, and recovering a purified 1-bromopropane product from the permanganate-containing 1-bromopropane product mixture.

In this invention, as used herein, the term “permanganate-containing 1-bromopropane product mixture” denotes a 1-bromopropane product mixture with which permanganate has been mixed, but which 1-bromopropane product mixture has not been separated from the permanganate, i.e., permanganate, in one form or another, is present in the 1-bromopropane product mixture. Similarly, the term “purified 1-bromopropane product,” as used herein, denotes a 1-bromopropane product mixture that has been mixed with permanganate, and from which the permanganate has been separated. Usually and preferably, the purified 1-bromopropane product undergoes further purification to form a finished 1-bromopropane product; if the purified 1-bromopropane product is not further purified, the purified 1-bromopropane product is the finished 1-bromopropane product.

Another embodiment of this invention is a process for preparing a 1-bromopropane product mixture from 1-propanol and hydrogen bromide, characterized by mixing permanganate with the 1-bromopropane product mixture to form a permanganate-containing 1-bromopropane product mixture, and recovering a purified 1-bromopropane product from the permanganate-containing 1-bromopropane product mixture.

Still another embodiment of this invention is a composition comprising permanganate and a 1-bromopropane product mixture.

Yet another embodiment of this invention is a composition comprising a purified 1-bromopropane product which is a 1-bromopropane product mixture that has been contacted with permanganate, and from which the permanganate has been removed.

These and other embodiments and features of this invention will be still further apparent from the ensuing description and appended claims.

### FURTHER DETAILED DESCRIPTION OF THE INVENTION

The term ppm means parts per million (wt/wt), unless specifically stated otherwise herein.

It is believed, though this invention is not bound by any theory of mechanism, that permanganate oxidizes at least two of the acid-forming impurities often found in 1-bromopropane products, be they crude products or refined products containing acid-reducing additives. Impurities that seem to contribute to the acidity of prior art 1-bromopropane products include 1-propanol and propionaldehyde. It is believed that the permanganate oxidizes these impurities to propionic acid, which can be reduced or removed by washing the 1-bromopropane product mixture with an aqueous solution of an inorganic base; this aqueous base wash will also remove unreacted permanganate from the 1-bromopropane product mixture. Unreacted permanganate can also be removed or reduced by washing the permanganate-containing 1-bromopropane product mixture with water.

Preferred 1-bromopropane product mixtures used in this invention have a purity of at least about 90%, and more preferably a purity of at least about 95%, and still more preferably a purity of at least about 98%, the balance in each case being one or more impurities resulting from the process by which the 1-bromopropane product mixture was prepared.

Permanganate can be mixed with a 1-bromopropane product mixture at almost any point after formation of the 1-bromopropane product mixture. While permanganate can be mixed with a crude 1-bromopropane product mixture (i.e., prior to any purification of the 1-bromopropane product mixture), normally and preferably the crude 1-bromopropane product mixture is washed with water and/or with an aqueous solution of at least one inorganic base prior to mixing with permanganate. More than one wash of the 1-bromopropane product mixture, or other purification steps, such as distillation, may be carried out before mixing permanganate with the 1-bromopropane product mixture.

For the mixing of permanganate with a 1-bromopropane product mixture, permanganate can be added to the 1-bromopropane product mixture, the 1-bromopropane product mixture can be added to permanganate, or permanganate and the 1-bromopropane product mixture can be co-fed to a vessel or mixing zone. Preferably, permanganate is added to a 1-bromopropane product mixture.

Basic conditions are preferred for mixing the permanganate with a 1-bromopropane product mixture. The pH is preferably in range of about 8 to about 10; higher pH values are acceptable but not necessary. By “basic conditions” it is meant that when water or an aqueous solution is stirred with a 1-bromopropane product mixture, and the aqueous and organic phases are allowed to separate, the pH of the aqueous phase is above 7. Basic conditions are typically achieved by performing one or more washes of a 1-bromopropane product mixture with an aqueous solution of an inorganic base, preferably an alkali metal base, more preferably an alkali metal hydroxide. Mixing of permanganate with a 1-bromopropane product mixture under acidic conditions is acceptable, but not preferred.

A purified 1-bromopropane product is recovered from a permanganate-containing 1-bromopropane product mixture by separating the permanganate from the 1-bromopropane product mixture. The separation can be accomplished in various ways, including distillation and washing. Washing the permanganate-containing 1-bromopropane product mixture with water or an aqueous solution of an inorganic base is a preferred way to separate the permanganate to obtain a purified 1-bromopropane product. An advantage to operation under basic conditions is that a water wash or a wash with an aqueous solution of an inorganic base of the permanganate-containing 1-bromopropane product mixture will extract propionic acid from the permanganate-containing 1-bromopropane product mixture. Without wishing to be bound by theory, permanganate is thought to oxidize propanol and propionaldehyde to propionic acid; thus, washing the permanganate-containing 1-bromopropane product mixture with water or an aqueous solution of an inorganic base will remove propionic acid so formed (in addition to removing unconsumed permanganate). Further purification steps such as distillation or one or more additional washes with water and/or an aqueous solution of an inorganic base may be performed on the purified 1-bromopropane product.

In the processes of this invention, drying is normally the final step in the purification of a purified 1-bromopropane product, after all of the other purification steps, if any, have been performed. It is generally not desirable to mix permanganate with a 1-bromopropane product mixture after the drying step, because permanganate will remain in the finished 1-bromopropane product.

Generally, drying of a l-bromopropane product mixture pursuant to the processes of this invention is carried out by contacting the purified 1-bromopropane product with a drying agent. Suitable drying agents are well known in the art and include calcium chloride, magnesium sulfate, calcium sulfate, potassium carbonate, and the like. A preferred drying agent in the practice of this invention is calcium chloride. Preferably, drying continues until the water content of the purified 1-bromopropane product is less than about 100 ppm (wt/wt); more preferably, drying continues until the water content of the purified 1-bromopropane product is less than about 80 ppm (wt/wt). Often, the purified 1-bromopropane product after drying is a finished 1-bromopropane product.

In some preferred processes of the invention, a 1,2-epoxide is mixed with the purified 1-bromopropane product. The 1,2-epoxide can be mixed into a purified 1-bromopropane product before or after the drying step, or a portion of the 1,2-epoxide can be mixed with the purified 1-bromopropane product before the drying step, and another portion (the remainder) of the 1,2-epoxide can be mixed with the purified 1-bromopropane product after the drying step. The purified 1-bromopropane product, after drying and inclusion of a 1,2-epoxide, is usually a finished 1-bromopropane product.

In the practice of this invention, permanganate, an anion, is mixed with a 1-bromopropane product mixture as one or more of its salts. Suitable permanganate salts include lithium permanganate, sodium permanganate, potassium permanganate, rubidium permanganate, cesium permanganate, calcium permanganate, magnesium permanganate, zinc permanganate, silver permanganate, lanthanum permanganate, and mixtures of any two or more of the foregoing. Preferred permanganate salts are sodium permanganate and potassium permanganate; especially preferred is potassium permanganate because of its ready availability and low cost. The permanganate salt(s) can be mixed with a 1-bromopropane product mixture in solid form or as a pre-formed solution of the permanganate salt.

Typically, permanganate is mixed with or contacted with a 1-bromopropane product mixture in the practice of this invention in proportions of about 0.1 to about 5 parts by weight permanganate per 1000 parts by weight of 1-bromopropane product mixture. Preferably, permanganate is mixed with a 1-bromopropane product mixture in proportions of about 0.25 to about 3 parts by weight permanganate per 1000 parts by weight of 1-bromopropane product mixture; more preferably, permanganate is mixed with a 1-bromopropane product mixture in proportions of about 0.75 to about 2.5 parts by weight of permanganate per 1000 parts by weight of 1-bromopropane product mixture. Larger amounts of permanganate can be used in the practice of this invention, but are not particularly desired, as excellent results are achieved with the amounts just described.

In the practice of the invention, when a permanganate salt is mixed with a 1-bromopropane product mixture as a pre-formed solution, normally and preferably an aqueous solution, the permanganate salt concentration in the solution is typically in the range of about 0.01 N to about 1 N. Preferably, the concentration of the permanganate salt in the solution is about 0.025 N to about 0.5 N.

Various water-soluble inorganic bases may be used in the aqueous solution of inorganic base. Such inorganic bases include oxides, hydroxides, acetates, sulfates, carbonates, and bicarbonates of the alkali metals, alkaline earth metals, zinc, ammonium, and the like. Examples of suitable bases include sodium oxide, potassium oxide, magnesium oxide, calcium oxide, zinc oxide, ammonium hydroxide, lithium hydroxide, sodium hydroxide, potassium hydroxide, rubidium hydroxide, magnesium hydroxide, calcium hydroxide, barium hydroxide, zinc hydroxide, ammonium nitrate, ammonium sulfate, lithium acetate, lithium carbonate, sodium acetate, sodium bicarbonate, sodium carbonate, potassium acetate, potassium bicarbonate, potassium carbonate, potassium nitrite, potassium sulfite, rubidium carbonate, cesium acetate, cesium bicarbonate, magnesium carbonate, calcium carbonate, and zinc carbonate. Mixtures of two or more inorganic bases can be used. Alkali metal bases are preferred; more preferred are alkali metal hydroxides, and sodium hydroxide and potassium hydroxide are highly preferred as inorganic bases. Typically, the aqueous solution of inorganic base has in the range of about 15 to about 75 weight percent inorganic base, and the aqueous solution of inorganic base preferably has in the range of about 25 to about 55 weight percent inorganic base.

The term 1,2-epoxide does not mean that the ring must involve the carbon atoms in the 1- and 2-positions; instead this means that the epoxide (cyclic ether) has three atoms in the ring rather than 4 atoms in the ring. Examples of suitable 1,2-epoxides include alkylene oxides and/or cycloalkylene oxides of up to about 8 carbon atoms, including propylene oxide, butylene oxide, pentene oxide, hexene oxide, heptene oxide, octene oxide, cyclopentene oxide, cyclohexene oxide, methyl-1,2-cyclopentene oxide, and the like, or mixtures thereof. Preferably, the 1,2-epoxide is butylene oxide, irrespective of whether the butylene oxide is 1,2-epoxybutane or 2,3-epoxybutane or a mixture of both.

Typically, enough 1,2-epoxide is mixed with the purified 1-bromopropane product to make a concentration of 1,2-epoxide in the purified 1-bromopropane product in the range of about 10 to about 1000 ppm (wt/wt), and preferably in the range of about 100 to about 600 ppm (wt/wt). More preferably, the 1,2-epoxide concentration is in the range of about 250 to about 500 ppm (wt/wt); still more preferred is a 1,2-epoxide concentration in the purified 1-bromopropane product in the range of about 400 to about 500 ppm (wt/wt).

The use of a 1,2-epoxide is optional in the practice of this invention. Especially for long periods of storage and/or exposure to high temperature (e.g., outdoor summer temperatures), the presence of a 1,2-epoxide in a finished 1-bromopropane product is desirable, but absent such conditions, the permanganate treatment alone may be sufficient to keep the acidity of a finished 1-bromopropane product within desirable limits.

Preferred processes of the invention include those in which a 1,2-epoxide is mixed with a purified 1-bromopropane product in an amount to make a concentration of about 250 ppm to about 500 ppm, especially where the 1,2-epoxide is butylene oxide; preferably, permanganate is sodium permanganate or potassium permanganate. More preferably, the processes further comprise drying the purified 1-bromopropane product until the water content of the purified 1-bromopropane product is less than about 100 ppm.

Other preferred processes of the invention comprise those in which permanganate is mixed with the 1-bromopropane product mixture in proportions of about 0.1 to about 5 parts by weight permanganate per 1000 parts by weight of 1-bromopropane product mixture, and wherein a 1,2-epoxide is mixed with a purified 1-bromopropane product in an amount to make a concentration of about 250 ppm to about 500 ppm, where the 1,2-epoxide is butylene oxide; preferably, the permanganate is sodium permanganate or potassium permanganate. More preferably, these processes further comprise drying the purified 1-bromopropane product until the water content of the purified 1-bromopropane product is less than about 100 ppm.

Still other preferred processes of the invention comprise those in which permanganate is mixed with a 1-bromopropane product mixture in proportions of about 0.1 to about 5 parts by weight permanganate per 1000 parts by weight of 1-bromopropane product mixture, wherein the permanganate is sodium permanganate or potassium permanganate, and wherein a 1,2-epoxide is mixed with the purified 1-bromopropane product in an amount to make a concentration of about 250 ppm to about 500 ppm. More preferably, the processes further comprise drying the purified 1-bromopropane product until the water content of the purified 1-bromopropane product is less than about 100 ppm.

In especially preferred processes of this invention, only water, one or more aqueous solutions of at least one inorganic base, and permanganate are brought into contact with a 1-bromopropane product mixture. It is preferred that only water and one or more aqueous solutions of at least one inorganic base are brought into contact with the purified 1-bromopropane product.

During the mixing of permanganate with a 1-bromopropane product mixture, pursuant to this invention, a new composition of matter is formed, comprising permanganate and the 1-bromopropane product mixture; this composition is a permanganate-containing 1-bromopropane product mixture. It is to be understood that the composition encompasses permanganate in whatever form into which the permanganate may be transformed when combined with a 1-bromopropane product mixture, and the 1-bromopropane product mixture.

Other preferred compositions of the invention comprising permanganate and a 1-bromopropane product mixture include those in which permanganate is present in proportions of about 0.1 to about 5 parts by weight per 1000 parts by weight of the 1-bromopropane product mixture. Still more preferred compositions of the invention comprising pennanganate and a 1-bromopropane product mixture are those in which permanganate is present in proportions of about 0.25 to about 3 parts by weight per 1000 parts by weight of 1-bromopropane product mixture. In especially preferred compositions of the invention comprising permanganate and a 1-bromopropane product mixture, only permanganate and the 1-bromopropane product mixture are present in the composition.

After the mixing of permanganate with a 1-bromopropane product mixture and the subsequent removal of the permanganate in the processes of this invention, a purified 1-bromopropane product that has been contacted with permanganate is produced. This purified 1-bromopropane product which is a 1-bromopropane product mixture that has been contacted with permanganate and from which the permanganate has been removed, is a new composition. Preferably, the water content of the composition comprising a purified 1-bromopropane product that has been contacted with permanganate is less than about 100 ppm; more preferably, the water content of the composition is less than about 80 ppm.

Preferred compositions of the invention comprising a purified 1-bromopropane product include those in which a 1,2-epoxide is present in the composition in a concentration of about 250 ppm to about 500 ppm, and wherein the 1,2-epoxide is butylene oxide. More preferably, the water content of a composition in which a 1,2-epoxide is present is less than about 100 ppm. In especially preferred compositions of the invention comprising a purified 1-bromopropane product, only the purified 1-bromopropane product and at least one 1,2-epoxide are present in the composition; if a 1,2-epoxide is not present in the composition, it is preferred that only the purified 1-bromopropane product is present in the composition.

In addition to the purified 1-bromopropane product and an optional 1,2-epoxide, one or more other substances, although unnecessary, may optionally be present in the composition that comprises a purified 1-bromopropane product. Such additives include nitroalkanes (e.g., nitromethane, nitroethane, etc.), N-alkylmorpholines, amines, dioxanes, dioxolanes, and other known stabilizers for 1-bromopropane. Surfactants, dyes, and other non-stabilizer components may optionally be included in the composition, provided no such component contributes substantially to an increase in the acidity of the composition, especially to an acidity above 10 ppm after 30 days at 60° C.

The following examples are presented for purposes of illustration, and are not intended to impose limitations on the scope of this invention.

The 60° C. stability test (also sometimes called the acidity test) used in the following Examples was conducted as follows: A quantity of about 160 grams of the finished 1-bromopropane product to be tested was placed in a 4 fluid ounce (118 mL) Boston Round screw cap bottle. The Teflon® polymer-lined cap for the bottle was applied without excluding air from the free head space. The capped bottle was held in a 60° C. oven for 30 days, opening it once at 10 days to take a sample for analysis. The sample was then allowed to cool to room temperature before determining the acidity. The analysis for acidity involved shaking 80-120 grams of the cooled test sample with 30 to 50 mL of ultra pure water followed by phase separation and titration of the aqueous phase with 0.01 N NaOH to the phenolphthalein endpoint. This acidity analysis will show a positive result for HBr, as well as for carboxylic acids such as propionic acid.

The gas chromatography analyses of the finished 1-bromopropane products in the following Examples were performed on a Hewlett-Packard 5890 gas chromatograph equipped with a split injector, flame ionization detector and a 30M×0.53 mm×3 μm DB-624 capillary column operating at 35° C. The temperature was held at 35° C. for 8 minutes, then raised at 10° C./min. to 230° C., final time 3 minutes. The column head pressure was 4.5 psig (1.32×10⁵ Pa) and the total flow of He was 75 mL/minute. An injection volume of 0.5 μL of neat sample was used. The amounts of 2-bromopropane, propanol, propionaldehyde, and propionic acid reported in the Examples below were determined by gas chromatography (GC), and are based on GC area percent, unless otherwise stated.

In the following Examples, finished 1-bromopropane products were analyzed for the presence of KMnO₄ using inductively coupled plasma (ICP) analysis for manganese, and the water content of the finished 1-bromopropane products were determined by coulometric Karl Fisher analysis.

Comparative Example A illustrates a typical process for preparing 1-bromopropane products from propanol and HBr, and Table A shows the amounts of impurities in a finished 1-bromopropane product at the end of a process and after storage when the 1-bromopropane product mixture is not mixed with permanganate. Examples 1-3 show processes of the invention.

### COMPARATIVE EXAMPLE A

A finished 1-bromopropane product was prepared from 1-propanol and HBr as follows:
(1) 1-Propanol (9000 L) was charged to a reactor. A maximum HBr flow (200 m³·h⁻¹) to the reactor was started while the temperature of the cooled and agitated mixture in the reactor was kept at <45° C. After the reaction was complete, the mixture was cooled to 30° C. Water (300 L) was added and the mixture was agitated. Agitation was stopped, and the mixture was allowed to settle. The resultant organic and aqueous phases were separated; the aqueous phase was discarded.
(2) While agitating the organic phase (the 1-bromopropane product mixture), aqueous NaOH (48%, 500 L) and water (100 L) were added to the organic phase (1-bromopropane product mixture). Agitation was stopped, the layers were allowed to separate, and the aqueous layer was discarded.
(3) Distillation of the organic layer (12500 kg) was started; the first 300 liters collected were discarded. The distilled organic layer was made to flow into water (1000 kg) already being stirred, until the temperature in the distillation column head reached 80° C. Aqueous NaOH (23%) was added to the mixture of distillate (12000 kg) and water (1000 kg) until the pH was between 8 and 10. The mixture was agitated. Once agitation was stopped, the mixture was allowed to settle. The resultant organic and aqueous phases were separated; the aqueous phase was discarded.
(4) Water was added to the organic phase, and the mixture was agitated. Aqueous NaOH (23 wt %) was added to the mixture until the pH was between 8 and 10. The mixture was agitated. Once agitation was stopped, the mixture was allowed to settle. The resultant organic and aqueous phases were separated; the aqueous phase was discarded.
(5) The organic phase was dried by passing it through a column containing CaCl₂ until the final water content of the 1-bromopropane product was less than 80 ppm. Enough butylene oxide was added to the 1-bromopropane product mixture to make a butylene oxide concentration of about 450-500 ppm.

No permanganate was added during or after the preparation. Results for the finished 1-bromopropane product initially as well as after 10 days and after 30 days at 60° C. are summarized in Table A, as amounts present in the finished 1-bromopropane product.

TABLE A

| | | Amount | Amount |
|---|---|---|---|
| | | present after | present after |
| | Amount present at | 10 days | 30 days |
| | start of stability test | at 60° C. | at 60° C. |
| 1-Bromopropane | 99.88% | 99.87% | 99.87% |
| 2-Bromopropane | 0.0161% | 0.0157% | 0.0159% |
| Propanol | 0.0175% | 0.0168% | 0.0163% |
| Acidity* | 5 ppm | 40 ppm | 60 ppm |
| Propionaldehyde | 11 ppm | 7 ppm | 10 ppm |
| Propionic acid | 0 ppm | 11 ppm | 25 ppm |

*Acidity is reported as ppm HBr.

A desirable variation on the process here described involves the addition of half of the desired amount of 1,2-epoxide to the 1-bromopropane product mixture prior to the distillation and the addition of the other half of the desired amount of 1,2-epoxide after the distillation of the 1-bromopropane product mixture.

### EXAMPLE 1

Step (1) of Comparative Example A was carried out to form a 1-bromopropane product mixture. Then an aqueous alkaline solution of KMnO₄ (421 g; 0.05 N) was added with stirring to the 1-bromopropane product mixture (2700 g). As a result of this operation, the amount of propanol present in the 1-bromopropane product mixture decreased from 100 ppm to 47 ppm, and no propionaldehyde remained (from an initial amount of propionaldehyde of 7 ppm) in the 1-bromopropane product mixture. The 1-bromopropane product mixture was then washed with aqueous caustic (120 g; 40 wt % NaOH), after which no more propionic acid was present in the 1-bromopropane product mixture. Distillation of the organic layer (2628 g) was started; the first 143 g collected were discarded. Distillation was continued until the temperature in the distillation column head reached 80° C. Aqueous NaOH (32 mL; 23 wt %) was added to the distillate (2233 g). This mixture was agitated for 1 hour. Once agitation was stopped, the mixture was allowed to settle. The resultant organic and aqueous phases were separated; the aqueous phase was discarded. Water (200 mL) was added to the organic phase, and the mixture was agitated. Aqueous NaOH (23 wt %) was added to the mixture until the pH was between 8 and 10. The mixture was agitated. Once agitation was stopped, the mixture was allowed to settle. The resultant organic and aqueous phases were separated; the aqueous phase was discarded. The organic phase was dried by passing it through a column containing CaCl₂ until the final water content was less than 80 ppm. Enough butylene oxide was added to the 1-bromopropane product mixture to make a butylene oxide concentration of about 450-500 ppm, after which the obtained finished 1-bromopropane product was subjected to the acidity test described above. Results for the finished 1-bromopropane product initially as well as after 10 days and after 30 days at 60° C. are summarized in Table 1, as amounts present in the finished 1-bromopropane product.

TABLE 1

| | | | Amount |
|---|---|---|---|
| | | Amount | present after |
| | Amount present at | present after | 30 days |
| | start of stability test | 10 days at 60° C. | at 60° C. |
| 1-Bromopropane | 99.9% | 99.9% | 99.9% |
| 2-Bromopropane | 181 ppm | 182 ppm | 183 ppm |
| Propanol | 23 ppm | 22 ppm | 23 ppm |
| Acidity | 1.8 ppm | 9.1 ppm | 3 ppm |
| Propionaldehyde | 0 ppm | 0 ppm | 0 ppm |
| Propionic acid | 0 ppm | 0 ppm | 0 ppm |

*Acidity is reported as ppm HBr.

It can be seen from Table 1 that propionaldehyde and propionic acid are not formed in detectable amounts during the stability test in a finished 1-bromopropane product obtained from a permanganate-treated 1-bromopropane product mixture.

### EXAMPLE 2

A 1-bromopropane product mixture was prepared as described above in Comparative Example A. After step (3), the 1-bromopropane product mixture was washed with an aqueous alkaline solution of KMnO₄. 168 Grams of an aqueous alkaline solution of KMnO₄ (0.05 N) were used per 1150 g of 1-bromopropane product mixture. After the KMnO₄ wash, when the aqueous and organic phases separated, the upper layer was the aqueous phase. Steps (4) and (5) of the preparation were then carried out, including the addition of the butylene oxide. Results for the finished 1-bromopropane product initially as well as after 10 days and after 30 days at 60° C. are summarized in Table 2, as amounts present in the finished 1-bromopropane product.

TABLE 2

| | Amount | Amount present | Amount present |
|---|---|---|---|
| | present at start | after 10 days | after 30 days |
| | of stability test | at 60° C. | at 60° C. |
| 1-Bromopropane | 99.87% | 99.83% | 99.84% |
| 2-Bromopropane | 0.0139% | 0.0139% | 0.0140% |
| Propanol | 0 ppm | 0 ppm | 0 ppm |
| Acidity* | 2.3 ppm | 3.0 ppm | 3.2 ppm |
| Propionaldehyde | 0 ppm | 0 ppm | 0 ppm |
| Propionic acid | 0 ppm | 0 ppm | 0 ppm |

*Acidity is reported as ppm HBr.

### EXAMPLE 3

Three runs were performed, each with a different amount of KMnO₄. A 1-bromopropane product mixture was prepared according to preparation in Comparative Example A. The KMnO₄ was added after step (3). To mix the 1-bromopropane product mixture with the solid KMnO₄, the 1-bromopropane product mixture (1150 g) was added to water (88.5 g). Aqueous NaOH (23 wt %) was added until the pH was between 8 and 10. Once this pH was obtained, solid KMnO₄ was added, and the mixture was stirred during 3 hours. The organic phase was extracted. Referring again to the preparation above, step (4) was omitted; step (5) was carried out, including the addition of butylene oxide. Results for the finished 1-bromopropane products initially as well as after 10 days and after 30 days at 60° C. are summarized in Table 3, as amounts present in the finished 1-bromopropane product.

While the acidity in Runs 1 and 2 does increase above 10 ppm by the 30th day at 60° C., the acidity in these two Runs is much less than that observed in 1-bromopropane product mixtures that have not been treated with permanganate (see Comparative Example A, above).

TABLE 3

| | Amount | Amount | Amount |
|---|---|---|---|
| | present at | present | present |
| | start of stability | after 10 days at | after 30 days at |
| | test | 60° C. | 60° C. |
| Run 1 | | | |
| (0.24 g KMnO₄ per kg | | | |
| 1-bromopropane) | | | |
| 1-Bromopropane | 99.88% | 99.87% | 99.87% |
| 2-Bromopropane | 0.0138% | 0.0139% | 0.0140% |
| Propanol | 0 ppm | 0 ppm | 0 ppm |
| Acidity* | 4 ppm | 11.5 ppm | 18.6 ppm |
| Propionaldehyde | 0 ppm | 0 ppm | 3 ppm |
| Propionic acid | 0 ppm | 0 ppm | 0 ppm |
| Run 2 | | | |
| (0.8 g KMnO₄ per kg | | | |
| 1-bromopropane) | | | |
| 1-Bromopropane | 99.91% | 99.85% | 99.87% |
| 2-Bromopropane | 0.0121% | 0.0126% | 0.0121% |
| Propanol | 0 ppm | 0 ppm | 0 ppm |
| Acidity* | 4 ppm | 21 ppm | 30.2 ppm |
| Propionaldehyde | 0 ppm | 2 ppm | 3 ppm |
| Propionic acid | 0 ppm | 0 ppm | 0 ppm |
| Run 3 | | | |
| (1.6 g KMnO₄ per kg | | | |
| 1-bromopropane) | | | |
| 1-Bromopropane | 99.92% | 99.90% | 99.90% |
| 2-Bromopropane | 0.014% | 0.0139% | 0.0140% |
| Propanol | 0 ppm | 0 ppm | 0 ppm |
| Acidity* | 3 ppm | 4.4 ppm | 3 ppm |
| Propionaldehyde | 0 ppm | 0 ppm | 0 ppm |
| Propionic acid | 0 ppm | 0 ppm | 0 ppm |

*Acidity is reported as ppm HBr.

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation in its practice.

1. A process which comprises mixing permanganate with a 1-bromopropane product mixture to form a permanganate-containing 1-bromopropane product mixture, and recovering a purified 1-bromopropane product from said permanganate-containing 1-bromopropane product mixture.

2. A process for preparing a 1-bromopropane product mixture from 1-propanol and hydrogen bromide, characterized by mixing permanganate with said 1-bromopropane product mixture to form a permanganate-containing 1-bromopropane product mixture, and recovering a purified 1-bromopropane product from said permanganate-containing 1-bromopropane product mixture.

3-5. (canceled)

6. A process as in claim 1 wherein said permanganate is sodium permanganate or potassium permanganate.

7. (canceled)

8. A process as in claim 1 wherein said permanganate is mixed with said 1-bromopropane product mixture in proportions of about 0.1 to about 5 parts by weight permanganate per 1000 parts by weight of 1-bromopropane product mixture.

9-10. (canceled)

11. A process as in claim 1 wherein the purified 1-bromopropane product is washed at least once with water or with an aqueous solution of at least one inorganic base.

12-15. (canceled)

16. A process as in claim 1 wherein only water, one or more aqueous solutions of at least one inorganic base, and permanganate are brought into contact with the 1-bromopropane product mixture.

17-20. (canceled)

21. A process as in claim 1 wherein said process further comprises drying the purified 1-bromopropane product until it has a water content of less than about 100 ppm (wt/wt).

22. (canceled)

23. A process as in claim 1 wherein at least one 1,2-epoxide is mixed with the purified 1-bromopropane product.

24. A process as in claim 23 wherein said 1,2-epoxide is butylene oxide.

25. A process as in claim 23 wherein said 1,2-epoxide is mixed in an amount to make a concentration of 1,2-epoxide in the purified 1-bromopropane product in the range of about 100 ppm to about 600 ppm (wt/wt).

26-29. (canceled)

30. A process as in claim 1 wherein said permanganate is sodium permanganate or potassium permanganate, wherein said permanganate is mixed with said 1-bromopropane product mixture in proportions of about 0.1 to about 5 parts by weight permanganate per 1000 parts by weight of 1-bromopropane product mixture, wherein said mixing is under basic conditions, and wherein said basic conditions involve a pH in the range of about 8 to about 10.

31-33. (canceled)

34. A process as in claim 1 further comprising forming a finished 1-bromopropane product from said purified 1-bromopropane product, wherein the finished 1-bromopropane product has an acidity of less than about 10 ppm as ppm HBr after 30 days at 60° C.

35. A process as in claim 1 wherein the 1-bromopropane product mixture has a purity of at least about 90%.

36. A process as in claim 1 wherein the 1-bromopropane product mixture has a purity of at least about 95%.

37-39. (canceled)

40. A composition comprising permanganate and a 1-bromopropane product mixture.

41. A composition as in claim 40 wherein permanganate is present in proportions of about 0.1 to about 5 parts by weight permanganate per 1000 parts by weight of 1-bromopropane product mixture.

42. (canceled)

43. A composition as in claim 40 wherein the 1-bromopropane product mixture has a purity of at least about 90%.

44. (canceled)

45. A composition as in claim 40 wherein only the 1-bromopropane product mixture and permanganate are present in the composition.

46. A composition comprising a purified 1-bromopropane product which is a 1-bromopropane product mixture that has been contacted with permanganate, and from which said permanganate has been removed.

47. A composition as in claim 46 wherein a 1,2-epoxide is present, and wherein said 1,2-epoxide has a concentration in said composition of about 250 ppm to about 500 ppm.

48. A composition as in claim 47 wherein the 1,2-epoxide is butylene oxide.

49. (canceled)

50. A composition as in claim 46 wherein the composition has a water content of less than about 100 ppm.

51. (canceled)

52. A composition as in claim 46 wherein the composition has an acidity of less than about 10 ppm as ppm HBr after 30 days at 60° C.

53. A composition as in claim 46 wherein only the purified 1-bromopropane product and at least one 1,2-epoxide are present in said composition.

54. A composition as in claim 46 wherein only the purified 1-bromopropane product is present in said composition.

55. (canceled)
